(19) United States
(12) Patent Application Publication
(10) Pub. No.: US 2022/0331370 A1
(43) Pub. Date: Oct. 20, 2022

(54) TREATMENT OF CARDIOVASCULAR DISEASES

(71) Applicant: Stemcyte Inc., Baldwin Park, CA (US)

(72) Inventors: Jonas Wang, Baldwin Park, CA (US); Shinn Zong Lin, Hualien (TW); Horng-Jyh Harn, Hualien (TW); Po-Cheng Lin, New Taipei City (TW)

(73) Assignee: Stemcyte Inc., Baldwin Park, CA (US)

(21) Appl. No.: 17/636,043

(22) PCT Filed: Aug. 20, 2020
(86) PCT No.: PCT/US20/47094

§ 371 Date: Feb. 17, 2022

Related U.S. Application Data
(60) Provisional application No. 62/889,225, filed on Aug. 20, 2019.

(51) Int. Cl. A61K 35/51; A61K 47/20; A61K 9/00; A61P 25/28; A61P 9/10

(52) U.S. Cl. CPC A61K 35/51; A61K 47/20; A61K 9/0019; A61P 25/28; A61P 9/10

(57) ABSTRACT

This invention relates to cell-based treatment of cardiovascular disease. The invention also provides treatments to improve neural tissue and to improve behavior and neurological function in cardiovascular disease patients as well as patients suffering from other forms of neurological stress or damage.

### CROSS REFERENCE TO RELATED APPLICATION

This application claims priority to U.S. Provisional Application No. 62/889,225 filed on Aug. 20, 2019. The content of the application is incorporated herein by reference in its entirety.

### FIELD OF THE INVENTION

This invention relates to cell-based treatment of cardiovascular diseases, such as stroke and cardiomyopathy, in patients. The invention also provides treatments to improve neural tissue and to improve behavior and neurological function in cardiovascular disease patients as well as patients suffering from other forms of neurological stress or damage.

### BACKGROUND OF THE INVENTION

Cardiovascular disease (CVD) is a class of diseases that involve the heart or blood vessels. CVD includes coronary artery diseases (CAD) such as angina and myocardial infarction (commonly known as a heart attack). Other CVDs include stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis. Cardiovascular diseases are the leading cause of death globally. For example, stroke is the second leading cause of death and physical disability. While one in six people in the world is affected by stroke, every 40 seconds, someone in the U.S. is having stroke. The cost of stroke care in many countries exceeds five percent of the healthcare budget (Mukherjee et al., Neurosurg. 2011, 76, S85-S90). Due to an aging population, stroke presents an even greater health and economic burden worldwide. Whereas a small stroke may lead to only minor problems, such as arm or leg weakness, a larger stroke may result in paralysis on one side or the inability to speak. More than two-thirds of stroke patients do not recover completely from a stroke even if they receive optimal care during the acute stroke phase (Hacke et al., Lancet 2004, 363, 768-774). Currently the only available approved pharmacological treatment for ischemic stroke is tissue plasminogen activator (TPA). However, relatively few patients are eligible for this therapy. There is a need for new therapies for treating cardiovascular disease.

### SUMMARY OF INVENTION

This invention addresses the need mentioned above in a number of aspects.

In one aspect, the invention provides a method of treating or ameliorating a cardiovascular disease or brain injury. The method comprises identifying a subject in need thereof and administering to the subject an effective amount of a therapeutic composition comprising umbilical cord blood (UCB) or cord blood (CB).

The therapeutic composition can comprise plasma-depleted (PD) UCB or red cell-reduced (RCR) UCB. In some embodiments, the therapeutic composition can further comprise a cryoprotectant, such as dimethyl sulfoxide (DMSO). The PD UCB is not depleted in red blood cells when compared to whole blood UCB. For example, the PD UCB can comprise at least 50% red blood cells by volume, or comprise all red blood cells by volume. The therapeutic composition can comprise from about 5% to 15% (e.g., 5-10%) cryoprotectant by volume. The composition can be obtained by thawing a stored, frozen composition comprising UCB and the cryoprotectant mentioned above. The step of thawing can be completed within 1 to 10 minutes, such as 5 minutes. In one example, the step of thawing comprises incubating the stored composition in a bath maintained at between about 37° C. and about 41° C., preferably, between about 37° C.±2° C. Once thawed, the stored composition need not be washed and can be administered directly as the therapeutic composition to the subject. The step of administering can be completed within 1 to 2 hours after the thawing is completed. The UCB comprises mononucleated cells and the mononucleated cells are administered to the subject at approximately 1×10⁶ cells/kg to approximately 1×10⁸ cells/kg. In one example, the mononucleated cells are administered at approximately 2-5×10⁸ mononucleated cells/kg to approximately 1×10⁹ cells/kg. The therapeutic composition can be administered by infusion at about 1-20 ml/minute, such as about 5-10 ml/minute. The method can further comprise administering a blood-brain barrier (BBB) permeabilizer composition to the subject. Examples of the BBB permeabilizer comprise mannitol.

The cardiovascular disease can be a stroke or cardiomyopathy. Examples of the stroke include an ischemic stroke, a hemorrhagic stroke, an acute stroke, or a sub-acute stroke. In preferred embodiments, the subject is a human patient. Before the administering step, the patient can have a National Institutes of Health Stroke Scale (NUBS) score of 4 to 32 or higher, such as 6-18 or 8-16. Examples of the cardiomyopathy include ischemic cardiomyopathy.

The method can comprise HLA typing the recipient-subject. Preferably, the recipient-subject and a selected cord blood unit or units of a multiple cord blood transplant, should be 4/6 or better HLA match. The method may or may not comprise ABO typing the recipient-subject as ABO blood group incompatibility between a recipient-subject and a donor has not been reported as an issue in cord blood transplantation. That is, ABO match or compatibility between a recipient-subject and a donor is not required.

The method described herein can further comprise administering the subject an immunosuppression agent, such as hydrocortisone. Preferably, the subject is not administered with a fibrinolytic drug, such as tissue plasminogen activator, before, during or after administering the cells as such a fibrinolytic drug may damage the administered cells.

This disclosure also provides use of the above-described therapeutic composition in the manufacture of a medicament for treating cardiovascular disease or brain injury.

The details of one or more embodiments of the invention are set forth in the description below. Other features, objectives, and advantages of the invention will be apparent from the description and from the claims.

### BRIEF DESCRIPTION OF THE DRAWINGS

FIG. 1 is a diagram showing a timeline for a clinical study of a stroke patient.

FIG. 2 is a diagram showing combination mannitol administration after a UCB infusion.

FIG. 3 is a diagram showing motor function improvement by the patient after receiving UCB infusion.

FIGS. 4A, 4B, and 4C are diagrams showing results of neurological functions from 1-day pre-CB transfusion (baseline) to 12 months post-CB transfusion. (A) NIHSS, (B) Berg balance score, and (C) Barthel index score.

FIGS. 5A, 5B, 5C and 5D are photographs showing results of diffusion weighted imaging (DWI) examined from 2 hours after stroke to 6 months post-CB transfusion. (a) 2 hours after stroke. (b) 1 day post-CB transfusion. (c) 3 months post-CB transfusion. (d) 6 months post-CB transfusion.

FIGS. 6A, 6B, 6C, and 6D are photographs showing T2 images examined from 2 hours after stroke to 6 months post-CB transfusion. (a) 2 hours after stroke. (b) 1 day post-CB transfusion. (c) 3 months post-CB transfusion. (d) 6 months post-CB transfusion.

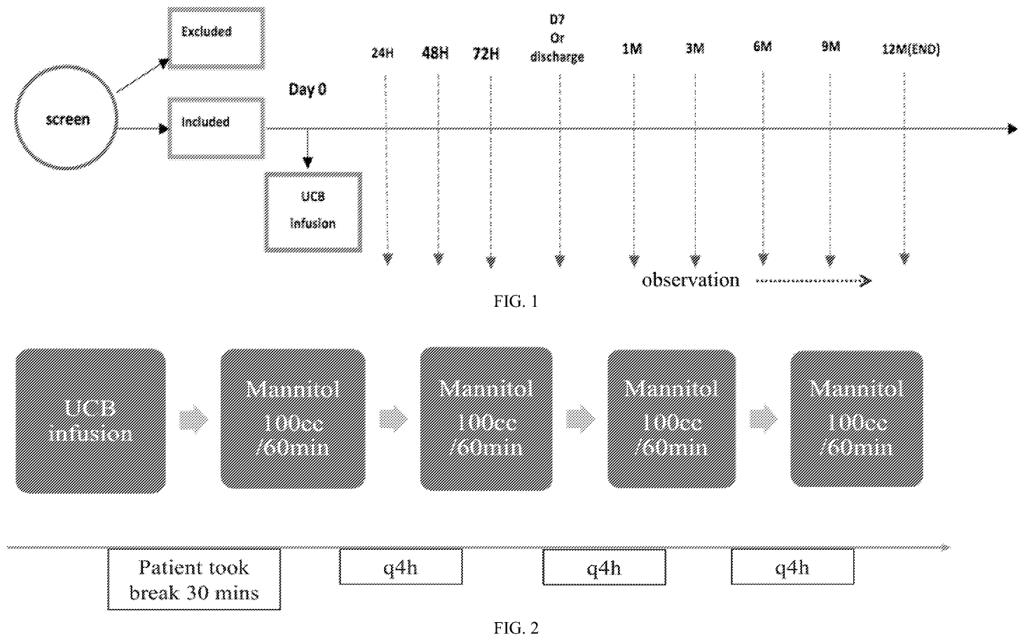

### DETAILED DESCRIPTION OF THE INVENTION

This invention provides methods and compositions to treat cardiovascular diseases, such as stroke and cardiac diseases. Certain aspects of this invention are based, at least in part, on an unexpected discovery that allogenic UCB units are safe and effective to treat patients with acute ischemic stroke. As disclosed herein, the UCB units possess not only stem cells but also superior immune-tolerance and immunomodulatory activities and high levels of factors such as EGF, VEGF, G-CSF, and IL-10. Thus, infusion of the UCB products may not only restore immune homeostasis but also facilitate brain repairing in acute stroke patients.

### Stroke

Stroke is the second leading cause of death and third leading cause of physical disability, affecting one in six people and presenting a great health and economic burden worldwide. More than 15 million people suffered stroke each year. Of these people, 30-35% die and near 75% of the survival sustain permanent disability

A stroke occurs when a blood vessel that carries oxygen and nutrients to the brain is either blocked by a clot (called an ischemic stroke) or by a blood vessel rupturing and preventing blood flow to the brain (called a hemorrhagic stroke). When that happens, part of the brain cannot get the blood and oxygen it needs, resulting death of brain cells. About 80 percent of strokes are ischemic strokes. Ischemic strokes occur when the arteries to one's brain become narrowed or blocked, causing severely reduced blood flow (ischemia). Non-contrast computed tomography (CT) is the primary imaging modality for the initial evaluation of patients with suspected stroke. Three main stages are used to describe the CT manifestations of stroke: acute (less than 24 hours), subacute (24 hours to 5 days) and chronic (weeks). Birenbaum et al., West J Emerg Med. 2011 February; 12(1): 67-76.

Current treatments in the acute phase include anticoagulant, antiplatelet, and thrombolytic agents. The use of such thrombolytic agents must be within three hours of stroke, and at most, it is only effective for six hours. Furthermore, a thrombolytic agent increases the hemorrhage rate by 15-20%. After ischemic stroke, about 120 million neurons die every hour, which is equivalent to 3.6 years of aging in brain function (Lakhan SE et al., Journal of Translational Medicine. 2009; 7:97). Furthermore, as dead neurons cannot regenerate, finding a way to regenerate neurons as well as other cells is critical. Cell therapy provides a major breakthrough in the treatment of stroke. For example, the inventor team used G-CSF injection combined with autologous hematopoietic stem cells (CD34+) brain transplantation for the treatment of 15 chronic ischemic stroke patients. The results confirmed its feasibility and safety. However, the results also indicated that the patient's cell age has a significant effect on the degree of improvement. For instance, proliferation and differentiation abilities of stem cell from an older individual (over 60 years old) are not as good as those of stem cells from a younger one.

As disclosed herein, a phase I clinical study showed a remarkable treatment progress: allogeneic PD-Umbilical Cord Blood infusion is safe and effective for treating a patient with stroke.

### Cardiac Diseases

Cardiac disease or heart disease is a disease for which several classes or types exist (e.g., Ischemic Cardiomyopathy (ICM), Dilated Cardiomyopathy (DCM), Aortic Stenosis (AS)) and, many require unique treatment strategies. Thus, heart disease is not a single disease, but rather a family of disorders arising from distinct cell types (e.g., myocardial cells) by distinct pathogenetic mechanisms. As used herein, cardiac disease encompasses the following non-limiting examples: heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or a combination thereof.

Some embodiments disclosed herein relate to a method for treating a cardiac disease in a subject. The method includes administering or providing to the subject a therapeutic composition comprising umbilical cord blood.

### Umbilical Cord Blood

Umbilical cord blood (or cord blood), blood that remains in the placenta and in the attached umbilical cord after childbirth, is routinely collected because it contains stem cells, which can be used to treat hematopoietic and genetic disorders. UCB is composed of all the elements found in whole blood, that is, red blood cells, white blood cells, plasma, and platelets.

As disclosed herein, volume reduced cord blood samples are used. Two methods are currently used to reduce cord blood samples and store UCB units, the red cell-reduced (RCR) method and the plasma-depleted (PD) method. Developed in the early 1990s (Proc. Natl. Acad. Sci. USA 92(22):10119-10122; 1995), the RCR method centrifuges the cord blood in a hyperosmotic solution (hetastarch or albumen), removing all but 21 ml of the cells and fluids, and adds 4 ml of dimethyl sulfoxide (DMSO). The PD method, developed at Stemcyte, removes plasma but retains all the cells. PD units are typically 80-120 ml/unit in volume compared to 25 ml/unit of RCR units. See U.S. Pat. No. 8,048,619 and Biol. Blood Marrow Transplant. 13(11):1346-1357; 2007. While each method and UCB thus prepared has its pros and cons (Young et al., Cell Transplantation, Vol 23, pp. 407-415, 2017), both can be used to practice the invention disclosed herein.

To collect and store UCB units, blood banks add DMSO to cord blood to protect cells during freezing. DMSO reduces ice formation inside cells and allows >90% of cells to survive freezing. However, >1% DMSO is toxic to blood cells at body temperature (37° C.). For that reason, it is standard in the art that care must be taken to minimize DMSO administration to patients, and DMSO is added to cord blood just before freezing and removed shortly after thawing so that the cells are not exposed to 1% for periods exceeding 30 min. If cord blood is exposed to >1% DMSO for 30 min or more, cord blood cells will die and clump together. This may result in emboli to the heart, chest pain, and other symptoms when cord blood is infused intravenously. Young et al., Cell Transplantation, Vol 23, pp. 407-415, 2017. Yet, as disclosed herein, it was unexpected that a PD-UCB unit, without removing DMSO in it, was safe and effective to treat a patient with acute ischemic stroke.

### PD-UCB

The PD-UCB compositions of the present invention possess the unique features of having plasma that is substantially depleted from the UCB unit and red blood cells (RBC) that are not depleted from the UCB unit. Such UCB units can be prepared by a process that combines plasma depletion with cryopreservation, selection, thawing, and/or transplantation of hematopoietic stem cells to provide superior clinical outcome by maximizing post-processing cell recovery and post-thaw infusion cell dose. In one example, a plasma-depleted, non-RBC-depleted UCB unit can be prepared by the process described in U.S. Pat. No. 8,048,619, the content of which is incorporated by reference in its entirety.

Briefly, a newborn's UCB is collected into a collection vessel such as a multi-bag blood collection bag containing an anticoagulant. The collection vessel typically contains from about 0.1 to about 100 ml of anticoagulant (e.g., about 1, 5, 10, 15, 20, 25, 30, 35, 40, 45, 50, 55, 60, 65, 70, 75, 80, 85, 90, 95, or 100 m1). Preferably, the collection vessel contains from about 23 ml to about 35 ml of anticoagulant. Non-limiting examples of anticoagulants include a citrate, phosphate, dextrose, and adenosine mixture (CPDA), a citrate, phosphate, and dextrose mixture (CPD), and an acid, citrate, and dextrose mixture (ACD). Preferably, the anticoagulant is CPDA, which can comprise 0.299% anhydrous citric acid, 0.263% dehydrate sodium citrate, 0.222% monobasic sodium phosphate (monohydrate), 3.19% dextrose, and 0.0275% adenosine. CPDA is isotonic and has a neutral pH, so the ratio of anticoagulant to blood is not critical. However, one skilled in the art will appreciate that the composition and/or volume of the anticoagulant in the collection vessel can depend upon the volume of cord blood collected from the donor. The collection bag can be weighed to determine the UCB collection volume by subtracting the collection bag weight with anticoagulant from the combined weight. The volume in the bag is determined by the volume of the UCB plus the volume of the anticoagulant.

The collected UCB can be delivered to a UCB processing laboratory, preferably within about 43 hours to allow for cryopreservation by about 48 hours after birth. However, cryopreservation up to about 72 hours after birth can also yield acceptable results.

The whole cord blood can be assayed at this point to determine the complete blood count. The pre-processing hematocrit (i.e., the percentage of red blood cells by volume) with anticoagulant typically has a range of from about 20% to about 60% for over 95% of the samples. The red blood cell concentration is usually between about 2 to about 10×10⁶/μ1 and the white blood cell concentration is usually between about 1 to about 30×10⁶/μl.

The UCB unit can be centrifuged, e.g., in a 3-bag collection blood bag, to separate the cellular fraction from the upper plasma fraction. The upper plasma portion can be removed into the second bag, which is then sealed off. In certain instances, if the remaining mostly cellular portion contains more than 60 cc, the product can be divided into two portions (e.g., in the original bag and in the third bag), each in its own collection/transfer bag. After plasma depletion, both the hematocrit (HCT) and the RBC concentration of the UCB unit increase about 1.2 to about 3 fold (average=about 1.6 to about 1.8 fold; median=about 1.7 to about 1.8-fold) relative to whole blood or red blood cell-depleted units.

The product in each collection/transfer bag can be then transferred via a sterile docking device to one freezing bag (e.g., CRYOCYTE bag). Typically, the UCB units can be cryopreserved in one freezing bag after plasma depletion and addition of pre-cooled (i.e., about 4° C.) cryoprotectant, e.g., with an approximate maximum volume of about 75 cc. However, some UCB units can be divided into two bags, e.g., with an approximate combined maximum volume of about 150 cc.

The plasma-depleted UCB/anticoagulant mixture can be then cooled to about 4° C. prior to the addition of one or more cryoprotectants. Typically, a cryoprotectant in the form of a solution can be added in an amount equal to about 25% to about 50% of the UCB/anticoagulant volume. For example, in instances where the UCB/anticoagulant volume in the plasma-depleted sample is 60 ml, the volume of cryoprotectant solution can be 15 ml. As a result, the UCB unit generally comprises about 5% to about 15% by volume of the cryoprotectant, e.g., about 5%, 6%, 7%, 8%, 9%, 10%, 11%, 12%, 13%, 14%, or 15% by volume of the cryoprotectant. In a preferred embodiment, the cryoprotectant solution comprises a mixture of about 50% DMSO and about 5% Gentran 40 (i.e., about a 10:1 ratio of DMSO to Gentran 40), to provide a final DMSO concentration of about 5% to about 10%. The DMSO/Gentran 40 cryoprotectant solution can be added to the UCB/anticoagulant mixture at a rate of about 0.75 ml per minute by a syringe pump with the freezing bag between ice packs on a rotator to achieve a final DMSO concentration of about 5% to about 10%. The plasma-depleted UCB units containing both anticoagulant and cryoprotectant can have a higher hematocrit (HCT) and the RBC concentration (i.e., at least about 1.6 fold) relative to whole blood or red blood cell-depleted units.

The plasma-depleted UCB mixture containing both anticoagulant and cryoprotectant can be then frozen and stored in the manner described in U.S. Pat. No. 8,048,619 and other methods known in the art. The processed UCB units can typically be stored below about −135° C., and preferably below about −150° C., in liquid nitrogen (e.g., liquid and/or vapor phase).

Prior to infusion or transplantation to a subject, the plasma-depleted UCB units are thawed as described herein. The thawed units are either washed or not washed prior to administration. Preferably, the thawed units are not washed. In certain instances, the thawed, non-washed units are administered to a patient by direct infusion. In certain other instances, the thawed, non-washed units are administered to a patient by infusion after the units have been diluted and/or reconstituted with an isotonic solution containing, for example, human serum albumin and Gentran (Dextran). As shown in the examples below, not washing plasma-depleted UCB units after thawing improves the clinical outcome in patient transplanted with such units. In fact, the cumulative incidence to platelet engraftment and the speed to engraftment for neutrophils and platelets are both increased relative to washed units, indicating that post-thaw washing actually delays or reduces the cumulative incidence of the engraftment of hematopoietic stem cells. Additionally, not washing plasma-depleted UCB units after thawing provides better recovery of nucleated cells, thereby increasing the total nucleated cell (TNC) dosage that is administered relative to washed units.

Compared with human adult peripheral blood, human umbilical cord blood contains richer hematopoietic primitive cells and numerous endothelial primitive cells, which have strong replication capacity in vitro and in vivo. As disclosed herein, samples of cryopreserved plasma depleted cord blood products were assayed to determine cytokine profiles using a R&D Human XL Cytokine Discovery 14 Plex panel. The results are shown in Table 1A. As shown in the table, the level of anti-inflammatory cytokine, IL-10, is significantly higher than that of pro-inflammatory cytokine such as IL-1-beta, IL-2, IL-6, IFN-gamma and TNF-alpha. The relatively higher levels of growth factors (GFs), such as EGF, FGF-basic, VEGF, G-CSF, and GM-CSF, were observed in comparison with those of cytokines, IL-1-beta, IL-2, IL-4, IL-5, IL-6, IFN-gamma and TNF-alpha. The high amounts of EGF, VEGF, G-CSF, and IL-10 in PD CB products allow inventors not only to restore immune homeostasis but also to enhance the repairing of the damaged brain nervous system in cerebral stroke patients.

TABLE 1A
Cytokine and Growth Factor Profiles in Cryopreserved
PD Cord Blood Products

| Cryopreserved PD | Concentration* | Total AMT (~75 ml)* |
|---|---|---|
| cord blood product | pg/mL | pg/unit |
| Cytokines | | |
| IL-1 beta | 14.96 + 4.45 | 1122 + 334 |
| IL-2 | 56.03 + 33.12 | 4202 + 2484 |
| IL-4 | 19.12 + 7.46 | 1434 + 559 |
| IL-5 | 55.53 + 15.61 | 4165 + 1171 |
| IL-6 | 60.57 + 16.37 | 4542 + 1228 |
| IL-8 | 277.12 + 259.34 | 20784 + 19450 |
| IL-10 | 144.02 + 69.66 | 10801 + 5224 |
| IFN-gamma | 38.98 + 8.42 | 2923 + 631 |
| TNF-alpha | 40.84 + 12.62 | 3063 + 946 |
| GM-CSF | 81.30 + 56.4 | 6097 + 4230 |
| Growth Factors | | |
| VEGF | 277.79 + 84.01 | 20834 + 6300 |
| G-CSF | 134.09 + 20.45 | 10056 + 1534 |
| EGF | 191.13 + 23.98 | 14335 + 1798 |
| FGF basic | 87.80 + 33.86 | 6584 + 2539 |

*Levels of cytokines and growth factors presented as mean + SD

TABLE 1B
Cytokine and Growth Factor Profiles in Adult Plasma/Serum

| | Concentration* |
|---|---|
| Adult blood plasma/serum | pg/mL |
| Cytokines | |
| IL-1 beta | 1.24 + 0.19 |
| IL-2 | 2.29 + 0.27 |
| IL-4 | 6.05 + 0.23 |
| IL-5 | 1.93 + 0.22 |
| IL-6 | 1.20 + 0.17 |
| IL-8 | 2.98 + 0.79 |
| IL-10 | 1.91 + 0.14 |
| IFN-gamma | 1.41 + 0.21 |
| TNF-alpha | 2.77 + 0.25 |
| GM-CSF | 4.46 + 0.15 |
| Growth Factors | |
| VEGF | 2.94 + 0.11 |
| G-CSF | 46.22 + 0.13 |
| EGF | 4.64 + 0.13 |
| FGF basic | 2.35 + 0.12 |

(J Cell Mol Med. 2018; 22: 6157-6166).

Moreover, cord blood stem cells have been found to proliferate and differentiate into neural cells and were effective in treatment for several neurodegenerative diseases. For cerebral stroke, intravenous injection of umbilical cord blood mononuclear cells can restore exercise ability and have neuroprotective functions. After transplantation of human umbilical cord blood mononuclear cells, levels of inflammatory factors (such as TNF-α, IL-1β, IL-2, etc.) were inhibited. In contrast, levels of inhibiters of inflammation (such as IL-10, TGF-β1, etc.) were increased. Consequently, after transplantation of human umbilical cord blood mononuclear cells, the anti-inflammatory processes achieve protective effect of neural cells. Besides having ant-inflammatory effects, umbilical cord blood mononuclear cells can spontaneously transfer to the damaged central nervous system and some to the spleen. Therefore, umbilical cord blood mononuclear cells can participate in the biosynthesis of lymphocytes and some studies have confirmed that lymphocytes were associated with neuroprotection in rats with acute stroke. A phase I clinical trial of human umbilical cord blood monocyte (HUCBM) in acute ischemic stroke through intravenous administration is carried out. This disclosure reports the first subject who nearly completely recovered from right hemiplegia 12 months after HUCBM therapy.

### Uses

The present invention provides compositions and methods to treat or ameliorate a cardiovascular disease, a brain injury or a neurodegenerative disorder in a subject. In one embodiment, the subject has disruption of the flow of blood in or around the brain. Preferably, the injury or disorder is cerebral ischemia. To that end, a therapeutic composition containing UCB cells described herein is administered systemically into a patient along with a BBB permeabilizer. The BBB permeabilizer can be administered to the subject before, after, or at the same time with the therapeutic composition.

Accordingly, within the scope of the invention is a therapeutic composition containing an effective amount of UCB cells and, optionally, an effective amount of a BBB permeabilizer. In one embodiment, the UCB cells are obtained from human umbilical cord blood and comprise a volume reduced cord blood sample. In a further embodiment, the cells comprise an effective amount of a mononucleated cell. In one embodiment, the composition is intended for systemic administration to an individual, although other methods for administration are contemplated.

The number of mononucleated cells administered, e.g., in a single dose, can be about, or at least, or more than, e.g., 1×10⁵, 1×10⁶, 1×10⁷, 1×10⁸, 1×10⁹, 1×10¹⁰, 1×10¹¹ cells per administration. In one embodiment the effective amount of the mononucleated cell is approximately 1×10⁷ to 1×10⁹ cells, more preferably is approximately 1×10⁸ to approximately 1×10⁹, such as approximately 2 to 5×10⁸. In another embodiment, the effective amount of the mononucleated cell is approximately 0.001 to 2×10⁸ cells/kg, such as 0.01 to 2×10⁸ cells/kg, 0.02 to 1×10⁸ cells/kg, and 0.5 to 5×10⁷ cells/kg.

The invention also provides a method to treat or ameliorate a cardiovascular disease, a brain injury or a neurodegenerative disorder in a subject. The method comprises administering an effective amount of UCB cells and an effective amount of a BBB permeabilizer to an individual with the condition or disease. In one embodiment, the UCB cells comprise a volume reduced cord blood sample. In a further embodiment, the cells comprise an effective amount of a mononucleated cell. In one embodiment, mononucleated cells can be frozen after being obtained from human umbilical cord blood and is thawed prior to administration to the subject.

For intravenous administration, a plurality of UCB cells can be delivered in, e.g., about, or no more than 10 mL, 20 mL, 30 mL, 40 mL, 50 mL, 60 mL, 70 mL, 80 mL, 90 mL, 100 mL, 150 mL, or 200 mL by intravenous infusion. The cells can be infused for any medically acceptable period of time. For example, the number of cells described above can be administered, e.g., infused, e.g., intravenously or intraarterially, over the course of about, or no more than, 15, 20, 25, 30, 35, 40, 45, 50, 55, or 60 minutes.

The UCB cells can be administered to an individual having a disruption in the flow of blood in or around the brain or CNS at any time after development of one or more symptoms of or neurological deficits, e.g., hypoxic injury or anoxic injury, attributable to, the individual's disruption in the individual. For example, the UCB cells can be administered within the first 21, 20, 19, 18, 17, 16, 15, 14, 13, 12, 11, 10, 9, 8, 7, 6, 5, 4, 3 or 2 days of development of the first symptom or neurological deficit exhibited by the individual. Preferably, the cells are administered within the first 8, 9, or 10 days of development of the first detectable symptom or neurological deficit in the individual. It is contemplated that treatment results in a decrease in cerebral infarct volume in comparison to the volume of a cerebral infarct in an untreated individual. In one embodiment, the volume is reduced by greater than approximately 10%, 15%, 20%, 25%, 30%, 35%, or 40%.

The above-described composition and method can be used to treat patients, including veterinary (non-human animal) patients, to alleviate the symptoms of a variety of pathological conditions for which cell therapy is applicable. For example, the cells of the present invention can be administered to a patient to alleviate the symptoms of neurological disorders or injuries such as cerebral ischemia or cerebral infarct; neurodegenerative diseases, such as Huntington's disease, Alzheimer's disease, and Parkinson's disease; traumatic brain injury; spinal cord injury; epilepsy; Tay Sach's disease; lysosomal storage diseases; amyotrophic lateral sclerosis; meningitis; multiple sclerosis and other demyelinating diseases; neuropathic pain; Tourette's syndrome; ataxia, drug addition, such as alcoholism; drug tolerance; drug dependency; depression; anxiety; and schizophrenia. In a preferred embodiment of the present invention, the cells are administered to alleviate the symptoms of stroke, cerebral ischemia or cerebral infarct.

In particular, the present invention is directed to a method of treating neurological damage in the brain or spinal cord which occurs as a consequence of genetic defect, physical injury, environmental insult or damage from a stroke, heart attack or cardiovascular disease (most often due to ischemia) in a patient. The method comprises administering an effective number or amount of UCB cells to the patient, wherein a BBB permeabilizer is co-administered to the patient. In one aspect of the present invention, UCB cells can be grafted into a patient's brain or spinal cord, for example, or may be administered systemically, such as, but not limited to, intra-arterial or intravenous administration.

### Treating Stroke

The compositions and methods of the present invention may be used for the treatment of stroke. Preferably, the compositions and methods are utilized from immediately following stroke, up until approximately 28 days after stroke (e.g., about 8, 9, or 10 days). In one preferred embodiment, the compositions and methods of the present invention are not limited in usage to the 3 hour post-stroke window that t-PA is limited to.

Stroke treatable according to the methods provided herein can be stroke attributable to any cause. In an embodiment, the stroke can be ischemic stroke. The ischemic stroke can be thrombotic stroke or embolic stroke. In another embodiment, the stroke can be due to systemic hypoperfusion, i.e., a reduction of blood flow to all parts of the body; or is due to venous thrombosis. In other embodiments, the ischemic stroke is caused by fibrillation of the heart, e.g., atrial fibrillation; paroxysmal atrial fibrillation; rheumatic disease; mitral or aortic valve disease; artificial heart valves; cardiac thrombus of the atrium or vertricle; sick sinus syndrome; sustained atrial flutter; myocardial infarction; chronic myocardial infarction together with ejection fraction of less than 28 percent; symptomatic congestive heart failure with ejection fraction of less than 30 percent; cardiomyopathy; endocarditis, e.g., Libman-Sacks endocarditis, Marantic endocarditis or infective endocarditis; papillary fibroelastoma; left atrial myxoma; coronary artery bypass graft surgery; calcification of the annulus of the mitral valve; patent foramen ovale; atrial septal aneurysm, left ventricular aneurysm without thrombus, isolated left atrial stroke on echocardiography without mitral stenosis or atrial fibrillation; and/or complex atheroma in the ascending aorta or proximal arch.

In another embodiment, the stroke can be hemorrhagic stroke. A hemorrhagic stroke can be caused by intra-axial hemorrhage (leakage of blood inside the brain). A hemorrhagic stroke can also be caused by extra-axial hemorrhage (blood inside the skull but outside the brain). In more specific embodiments, the stroke can be caused by intraparenchymal hemorrhage, intraventricular hemorrhage (blood in the ventricular system), epidural hematoma (bleeding between the dura mater and the skull), subdural hematoma (bleeding in the subdural space), or subarachnoid hemorrhage (between the arachnoid mater and pia mater). Most hemorrhagic stroke syndromes have specific symptoms (e.g., headache, previous head injury). In other more embodiments, the hemorrhagic stroke can be caused by or associated with hypertension, trauma, bleeding disorders, amyloid angiopathy, illicit drug use (e.g., amphetamines or cocaine), or a vascular malformation.

The method of treatment provided herein results in the elimination of, a detectable improvement in, a lessening of the severity of or a slowing of the progression of one or more symptoms of a disruption of blood flow in or around the brain or CNS or a neurological deficit attributable to a disruption of blood flow in or around the brain or CNS, e.g., stroke, e.g., causing hypoxic injury or anoxic injury. In specific embodiments, the symptoms or neurological deficits comprise hemiplegia (paralysis of one side of the body); hemiparesis (weakness on one side of the body); muscle weakness of the face; numbness; reduction in sensation; altered smell, taste, hearing, or vision; loss of smell, taste, hearing, or vision; drooping of eyelid (ptosis); weakness of ocular muscles; decreased gag reflexes; decreased ability to swallow; decreased pupil reactivity to light; decreased sensation of the face; decreased balance; nystagmus; altered breathing rate; altered heart rate; weakness in sternocleidomastoid muscle with decreased ability or inability to turn the head to one side; weakness in the tongue; aphasia (inability to speak or understand language); apraxia (altered voluntary movements); a visual field defect; a memory deficit; hemineglect or hemispatial neglect (deficit in attention to the space on the side of the visual field opposite the lesion); disorganized thinking; confusion; development of hypersexual gestures; anosognosia (persistent denial of the existence of a deficit); difficulty walking; altered movement coordination; vertigo; disequilibrium; loss of consciousness; headache; and/or vomiting.

The severity of disruption of the flow of blood in or around the brain or CNS, e.g., severity of stroke, or of stroke symptoms and/or neurological deficits attributable to stroke, can be assessed using one or more widely accepted neurological function scales. For example, a subject's neurological function can be assessed by one or more of the Modified Rankin Scale, NIH Stroke Scale, Canadian Neurological Scale (CNS), Glasgow Coma Scale (GCS), Hempispheric Stroke Scale, Hunt & Hess Scale, Mathew Stroke Scale, Mini-Mental State Examination (MMSE), Orgogozo Stroke Scale, Oxfordshire Community Stroke Project Classification (Bamford), Scandinavian Stroke Scale, Japan Coma Scale (JCS), Barthel Index and/or Japan Stroke Scale (JSS). In specific embodiments, the improvement is detectable within 1, 2, 3, 4, 5, or 6 days, or within 1, 2, 3, 4, 5, 6, 8, 9, 10, 11 or 12 weeks after initial assessment, and after one or more administrations of UCB cells.

As disclosed herein, cryopreserved cord blood mononuclear cells from healthy donors have a more stable quality to insure successful therapy. For instance, shown in the examples below are results from a phase 1 study with patients aged 45-80 years who sustained an acute ischemic stroke. A Plasma-Depleted processed umbilical cord blood unit was selected from StemCyte's public cord blood bank based on ABO/Rh blood type matched, HLA 6/6 matched, and total nucleated cells (e.g., ranging between 0.5-5×10⁷ cells/kg). The primary aims in the study included Adverse Event (AE) and serious AE during 12-month follow up and Graft Versus Host Disease (GVHD) within 100 days post-transfusion. The secondary aims included the change of NIHSS, Barthel index, and Berg balance scale. The results shown included those from a 46 year-old male who was enrolled with identical ABO/Rh, matched 6/6 HLA, and available 2.63×10⁸ mononucleated cells. No serious AE or GVHD was found through 12 months observation. The patient's NIHSS improved from 9 to 1 point, Berg balance scale from 0 to 48 point, and Barthel index from 0 to 90 point. These results demonstrated a nearly complete recovery of hemiplegia in the patient within 12 months of time after using allogeneic umbilical cord blood.

### Treating Cardiac Diseases

The compositions and methods of the present invention may be used for the treatment of a cardiac disease too. Preferably, the compositions and methods are utilized from immediately following onset of the disease.

Some embodiments of the disclosure relate to a method for promoting cardiac muscle regeneration in a subject. The method includes administering or providing to the subject a therapeutic composition as disclosed herein. In some embodiments, a method for treating a cardiac disease or promoting cardiac muscle regeneration in a subject as disclosed herein optionally includes a process of identifying or selecting the subject as having or suspected of having a cardiac disease. The process of identifying or selecting can be carried out prior to administration of one or more therapeutic compositions and therapeutic agents or therapies.

In some embodiments, the cardiac disease is myocardial infarction, ischemic heart disease, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary stenosis, pulmonary regurgitation, tricuspid stenosis, tricuspid regurgitation, congenital disorder, genetic disorder, or a combination thereof. In some particular embodiments, the cardiac disease is myocardial infarction. In some other particular embodiments, the cardiac disease is ischemic heart disease where cardiac muscle regeneration is required.

### BBB Permeabilizer

As used herein, the term “blood brain barrier permeabilizer” or “BBB permeabilizer” is a substance that is capable of disrupting the blood brain barrier. In one embodiment, the disruption is temporary. The amount of BBB permeabilizer administered with the UCB cells is the amount effective to disrupt the BBB and allow neurotrophic growth factors to enter the brain in increased amounts and/or allow the cells to enter the brain. In one embodiment, the BBB allows increased entry of neurotrophic factors or cells into the brain when measured with 0-10 days after administration.

Various BBB permeabilizers are known in the art. A BBB permeabilizer can be one selected from the group consisting of mannitol, Cereport, small fat-soluble molecules, glucose, amino acids, dihydroxyphenylalanine, choline, and purine bases and nucleosides or derivatives thereof. In one embodiment, the BBB permeabilizer is mannitol. In another embodiment, the BBB permeabilizer is Cereport. Mannitol can be administered at a concentration of approximately 0.1 M to approximately 10 M, e.g., approximately 0.5 to 5 M, or approximately 1.0 M to 2.0 M. In one embodiment, the concentration of mannitol is approximately 1.07 M (or 20%). Cereport can be administered at a concentration of approximately 1 μg/kg to approximately 50 μg/kg, e.g., approximately 5 to 20 μg/kg. In one embodiment, the concentration of Cereport is approximately 9 μg/kg.

While it is contemplated that the BBB permeabilizer is administered to the subject at approximately the same time as the UCB cells, the BBB may be administered in a separate composition from the cells. It is contemplated that the BBB permeabilizer may be administered prior to, simultaneously with, or after the administration of the cells. In addition, it is contemplated that the methods of the current invention may further comprise re-administering the BBB permeabilizer with or without the administration of further cells to the individual at approximately 1-72 hours after initial administration, or thereafter administered daily, weekly, monthly or yearly depending on the stroke outcome.

### Additional Therapeutic Agents

The treatment method descried herein can further comprise administration to the subject one or more second therapeutic agents. Such second therapeutic agents can be administered before administration of UCB cells, during administration of the cells, or after administration of the cells. The second therapeutic agents can be administered fewer, more, or an equal number of times as the cells are administered.

A second therapeutic agent can be any agent that has therapeutic benefit to an individual having a disruption of blood flow in or around the brain or CNS. In one embodiment, the therapeutic agent is an agent, e.g., a drug, that is used to treat stroke, hypoxic injury or anoxic injury. In a specific embodiment, the second therapeutic agent is a neuroprotective agent.

In one embodiment, in which the individual suffers from hemorrhagic stroke, the second therapeutic agent can be a therapeutic agent that lowers blood pressure of said individual. In some embodiments, the second therapeutic agent can be an antihypertensive drug, e.g., a beta blocker or diuretic drug, a combination of a diuretic drug and a potassium-sparing diuretic drug, a combination of a beta blocker and a diuretic drug, a combination of an angiotensin-converting enzyme (ACE) inhibitor and a diuretic, an angiotensin-II antagonist and a diuretic drug, and/or a calcium channel blocker and an ACE inhibitor. In another embodiment, the second therapeutic agent can be administered in order to reduce intracranial pressure. In a more specific embodiment, the second therapeutic agent can be a diuretic.

Additional therapeutic agents useful also include, but are not limited to, anti-platelet therapy, thrombolysis, primary angioplasty, heparin, magnesium sulphate, insulin, aspirin, cholesterol lowering drugs, and angiotensin-receptor blockers (ARBs). In particular, ACE inhibitors have clear benefits when used to treat patients with chronic heart failure and high-risk acute myocardial infarction; this is possibly because they inhibit production of inflammatory cytokines by angiotensin II. A non-limiting listing of additional therapeutic agents and therapies includes ACE inhibitors, such as Captopril, Enalapril, Lisinopril, or Quinapril; Angiotensin II receptor blockers, such as Valsartan; Beta-blockers, such as Carvedilol, Metoprolol, and bisoprolol; Vasodilators (via NO), such as Hydralazine, Isosorbide dinitrate, and Isosorbide mononitrate; Statins, such as Simvastatin, Atrovastatin, Fluvastatin, Lovastatin, Rosuvastatin or pravastatin; Anticoagulation drugs, such as Aspirin, Warfarin, or Heparin; or Inotropic agents, such as Dobutamine, Dopamine, Milrinone, Amrinone, Nitroprusside, Nitroglycerin, or nesiritide; Cardiac Glycosides, such as Digoxin; Antiarrhythmic agents, such as Calcium channel blockers, for example, Verapamil and Diltiazem or Class III antiarrhythmic agents, e.g., Amiodarone, Sotalol or, defetilide; Diuretics, such as Loop diuretics, for example, Furosemide, Bumetanide, or Torsemide, Thiazide diuretics, for example, hydrochlorothiazide, Aldosterone antagonists, for example, Spironolactone or eplerenone. Alternatively or in addition, other treatments of cardiac disease are also suitable, such as Pacemakers, Defibrillators, Mechanical circulatory support, such as Counterpulsation devices (intraaortic balloon pump or noninvasive counterpulsation), Cardiopulmonary assist devices, or Left ventricular assist devices; Surgery, such as cardiac transplantation, heart-lung transplantation, or heart-kidney transplantation; or immunosuppressive agents, such as Myocophnolate mofetil, Azathiorine, Cyclosporine, Sirolimus, Tacrolimus, Corticosteroids Antithymocyte globulin, for example, Thymoglobulin or ATGAM, OKT3, IL-2 receptor antibodies, for example, Basilliximab or Daclizumab are also suitable.

In embodiments in which the administered cells are not autologous to the subject being treated, the second therapeutic agent can be an immunosuppressive agent. Immunosuppressive agents are well known in the art and include, e.g., anti-T cell receptor antibodies (monoclonal or polyclonal, or antibody fragments or derivatives thereof, e.g., Muromonab-CD3), anti-IL-2 receptor antibodies (e.g., Basiliximab (SIMULECT) or daclizumab (ZENAPAX), azathioprine, corticosteroids, cyclosporine, tacrolimus, mycophenolate mofetil, sirolimus, calcineurin inhibitors, and the like. In a specific embodiment, the immunosuppressive agent can be corticosteroids or a neutralizing antibody to macrophage inflammatory protein (MIP)-1α or MIP-1β.

The therapeutic compositions, pharmaceutical formulations disclosed herein and the additional therapeutic agents or therapies can be formulated into final pharmaceutical preparations suitable for specific intended uses. In some embodiments, the therapeutic composition and the additional therapeutic agent or therapy can be administered in a single formulation. In some embodiments, each of the therapeutic composition and the additional therapeutic agent or therapy is administered in a separate formulation. In some embodiments of the methods disclosed herein, the therapeutic composition and/or the additional therapeutic agent or therapy can be administered to the subject in a single dose. In some embodiments, the therapeutic composition and/or the additional therapeutic agent or therapy can be administered to the subject in multiple dosages. In some embodiments, the dosages are equal to one another. In some embodiments, the dosages are different from one another. In some embodiments, the therapeutic composition and/or the additional therapeutic agent or therapy is administered to the subject in gradually increasing dosages over time. In some embodiments, the therapeutic composition and/or the additional therapeutic agent or therapy is administered in gradually decreasing dosages over time.

### Matching

In one aspect of the invention, matching of a donor cell source can be performed by assessment of the HLA disparity between the donor cells and the recipient. In certain aspects, transplantation of donor cells is performed only if the donor cell graft matches at 4 or more out of 6 HLA loci for HLA-A, HLA-B, and HLA-DRB 1.

In other embodiments of the invention, cells may be matched using standard HLA matching that is currently performed clinically. The degree of matching acceptable for cord blood can be 4/6 loci selected from HLA-A, HLA-B, and HLA-DRB1. HLA-A and HLA-B may be typed by means of the standard 2-stage complement-dependent microcytotoxicity assay, and antigens assigned as defined by the World Health Organization (WHO) HLA nomenclature committee. HLA-DRB1 type may be determined by hybridization of polymerase chain reaction (PCR)-amplified DNA with sequence-specific oligonucleotide probes (SSOPs), with sequencing if needed.

Accordingly, on embodiment of the invention involves administration of cord blood, or fractions thereof into a recipient that has been properly matched with either HLA 4/6 loci matching and/or mixed lymphocyte reaction matching.

The examples below illustrate exemplary protocols for thawing and direct infusion of the plasma-depleted, cryopreserved cord blood units of the present invention without performing any washing steps. The direct infusion method without washing potentially results in the least amount of cell loss due to the lack of a wash step and obviates the longer manipulations of reconstitution. Available clinical data from transplant centers using cord blood products from StemCyte have demonstrated that direct infusion can produce superior outcome compared to infusing products after wash despite of the greater amount of DMSO, red blood cells and lysed hemoglobin, which may create clinically significant events in some patients. Because the StemCyte cord blood products have not been reduced of red blood cells, they are unlike cord blood units from other banks; therefore, it is important to follow StemCyte protocols to ensure correct administration of the product.

Because the product is undiluted, and exposed to 10% DMSO that is highly cytotoxic, the time from thaw to infusion must be minimized, therefore infusion should be completed within 20 minutes from the beginning of thawing. During infusions, an IV gravity drip may not work properly due to the viscosity of the thawed product. An IV push with a large 60 cc) syringe and wide bore needle may be necessary to avoid harmful delays in infusion.

Because of potential adverse reactions that may be anticipated from the administration of any cryopreserved hematopoietic stem cell product containing significant quantities of DMSO and lysed and intact red blood cells which may be ABO/Rh incompatible, a treatment/transplant center should follow its own internal protocols for premedication, patient monitoring and intervention to treat any of the anticipated adverse reactions. A treatment/transplant center should follow its own internal protocols for post-thaw testing of the cord blood product, including but not limited to ABO/Rh typing, HLA typing, viability, CBC, CD34+ cell enumeration, clonogenic potential, etc. However, depending on the protocol used, CFU assays may not be optimized for enumeration of the true functional capacity of StemCyte cord blood units, which have higher cell density. Transplant centers can consult StemCyte or Stem Cell Technologies about protocols for CFU testing of post-thawed StemCyte cord blood units.

Attached segments may be used for HLA or ABO/Rh type identity testing, CFU, CD34+ and/TNC (total nucleated cell) counts, as well as microbial testing purposes. A sampling site coupler may also be inserted into either the CBU freezing bag or the final thawed/washed CBU bag for sample testing. The amount of cord blood removed for sampling is left to the discretion of the transplant facility. Dispose of the product/empty infusion bag as well as all supplies in contact with the cord blood product as biomedical waste according to federal, state, local and/or institutional requirements.

Adverse reactions can happen after cell infusion. Although not all of the side effects described herein may occur, if they do occur they may need medical attention from patients' doctors or nurses immediately.

Mild to Moderate:

Frequent: nausea, vomiting, hypertension, hypotension, bradycardia, hemoglobinuria, shivering, sweet cream corn or garlic taste (from DMSO expiration).

Less frequent: headache, abdominal cramps, diarrhea, flushing, chills, fever, flushing, chest tightness, vertigo, encephalopathy, seizure, bradycardia, hyperbilirubinemia, increased serum transaminase levels.

Severe to Life Threatening:

Extremely rare (˜0.4% in the largest published study of 1,410 patients) and usually self-limited:

Cardiac—bradycardia, heart block, arrhythmia, shock, and cardiac arrest.

Neurologic—encephalopathy (possibly related to greater than 2 g DMSO/kg recipient weight and treatable by plasmapheresis), seizure (possibly related to very high cellular concentration of greater than 3.7×108 nucleated cells/ml; StemCyte's products are usually at one-tenth of this concentration).

Pulmonary—Respiratory depression.

Immunologic—Anaphylactic reaction.

Renal—acute renal failure due to high concentration of free hemoglobin (mitigated by premedication with antihistamine and corticosteroid, adequate hydration, urinary alkalization, mannitol diuresis).

Causes of potential adverse reactions include the followings:

DMSO Toxicity:

Though the acute toxic dose of DMSO for humans has not been determined, the LD50 value (amount of DMSO required to kill 50% of the test animals) reported for IV administration of DMSO are between 3.1-9.2 g/kg for mice, 2.5/kg for dogs, and greater than 11 g per kg for monkeys (reference 10.14). Most published reports have kept the DMSO dose below 1 g/kg recipient weight (reference 10.8). In a typical StemCyte cord blood unit, the maximal DMSO dose is between 7.5 (1 bag) to 15.0 g (2 bags); therefore, in patients with compromised renal functions and in small patients (under 7 kg for 1 bag and under 15.0 kg if two bags are to be administered at the same time), where achieving an adequate cell dose is not a problem, washing of the product is strongly recommended. The recommended rate, according to the literature, of infusion of 10% DMSO cryopreserved stem cell product varies between 5 to 20 ml per minute, and we are recommending an infusion rate of 5 to 10 ml per minute.

Volume Overload:

It is strongly recommended that the maximal volume to be transfused should be in the range of 5-15 ml/kg/dose.

Major ABO blood group incompatibility:

Although ABO blood group incompatibility between patient and donor has not been reported as an issue in cord blood transplantation, the following additional information is supplied in cases of major blood group incompatibility, e.g., patient is blood group 0 and the cord blood unit is not group 0. Although transfusion of large volumes of major ABO incompatible RBCs is known to cause transfusion reactions in patients who have a significant titer of anti-A and/or anti-B, Bensinger et al (Transplantation 33:427-429 (1982)) stated that when the recipient's anti-A and anti-B hemagglutinin titers are 1:16 or less, entire units of ABO incompatible RBCs may be transfused safely. Sauer-Heilborn et al (Transfusion 44:907-916 (2004) describe experience with transfusion of ABO incompatible peripheral blood stem cell and marrow units and state that the risk is lower with PBSC units (RBC volume 75-100 ml) compared to BM components (RBC volume 300-400 ml). The volume of RBC in the StemCyte cord blood unit is about 40 to 100 ml (post processing). This volume of RBC is not known to cause serious adverse effects, but symptoms such as elevated temperature, increased pulse and muscle aching may occur in rare occasions. Dark or red urine and plasma are expected because of the hemolysis in the cord blood sample.

Additional published experience with infusing stem cell products containing ABO incompatible RBCs are presented below: Dinsmore et al (Br J Haematol. 1983; 54:441-449) reported that two patients who received 68 and 45 ml of RBCs, respectively, developed transient hemoglobinuria without renal impairment. Seven additional patients developed low-grade fever and ten additional patients experienced no adverse reaction. Warkentin et al Vox Sang. 1985; 48:89-104 reported that marrow products containing up to 21 ml of RBCs resulted in measurable evidence of hemolysis in some patients but no patient had a reaction that was judged to be clinically severe. Braine et al (Blood 1982; 60:420-425) reported symptomatic reactions in 10 of 25 patients after transfusion of units containing up to 38 ml of ABO incompatible RBCs. Symptoms included transient fever, hypertension, chills, hemoglobinuria, bradycardia and confusion.

It is recommended that the rate of infusion be about 5-10 ml/minute, with close patient monitoring. Sauer-Heilborn et al (Transfusion 2004; 44:907-916) recommend that the patient should be premedicated with antipyretics and/or antihistamines and should be well hydrated. Other investigators also pre-medicate with corticosteroids. Adverse reactions usually occur during the infusion and resolve after the infusion is stopped. However, some reactions can occur 6-7 hours after the completion of the infusion so that patients should be monitored throughout that time period. Significant adverse reactions must be reported to the cord blood bank.

Common premedication regimens include adequate hydration (especially with major ABO mismatch), antihistamines (especially with major ABO mismatch), corticosteroids, mannitol, antiemetics, and antipyretics (especially with major ABO mismatch). Common therapeutic interventions for adverse events include diuretics (volume overload), anticonvulsant (seizures), atropine (bradycardia), plasmapheresis (eencephalopathy), 02 (pulmonary depression), and nnarcotics.

### Definitions

The term “umbilical cord blood” or “UCB” is used herein to refer to blood obtained from a neonate or fetus, most preferably a neonate and preferably refers to blood that is obtained from the umbilical cord or the placenta of newborns. Preferably, the umbilical cord blood is isolated from a human newborn. The use of umbilical cord blood as a source of mononuclear cells is advantageous because it can be obtained relatively easily and without trauma to the donor. In contrast, the collection of bone marrow cells from a donor is a traumatic experience. Umbilical cord blood cells can be used for autologous transplantation or allogenic transplantation, when and if needed. Umbilical cord blood is preferably obtained by direct drainage from the cord and/or by needle aspiration from the delivered placenta at the root and at distended veins. As used herein, the term “umbilical cord blood cells” refers to cells that are present within umbilical cord blood. In one embodiment, the umbilical cord blood cells are mononucleated cells that are further isolated from the umbilical cord blood using methods known to those of skill in the art. In a further embodiment, the umbilical cord blood cells may be further expanded and/or differentiated prior to administration to a patient.

The terms “umbilical cord blood unit” and “UCB unit” as used herein refer to a volume of cord blood that is collected from a single donor. The UCB compositions of the present invention typically contain one UCB unit, but may also contain multiple UCB units, e.g., double cord blood units that can be administered to a patient in order to further increase cell dosage.

The term “cord blood stem cells” refers to a population enriched in hematopoietic stem cells, or enriched in hematopoietic stem and progenitor cells, derived from human umbilical cord blood and/or human placental blood collected at birth. The hematopoietic stem cells, or hematopoietic stem and progenitor cells, can be positive for a specific marker expressed in increased levels on hematopoietic stem cells or hematopoietic stem and progenitor cells, relative to other types of hematopoietic cells. For example, such markers can be CD34, CD43, CD45RO, CD45RA, CD59, CD90, CD109, CD117, CD133, CD166, HLA DR, or a combination thereof. In addition, the hematopoietic stem cells, or hematopoietic stem and progenitor cells, can be negative for an expressed marker, relative to other types of hematopoietic cells. For example, such markers can be Lin, CD38, or a combination thereof. In particular embodiments, the hematopoietic stem cells, or hematopoietic stem and progenitor cells, are CD34+ cells.

The term “stem cell” refers to any cell that has the ability to divide for indefinite periods of time and to give rise to specialized cells. Stem cells emanate from all germinal layers (i.e., ectoderm, mesoderm, and endoderm). Typical sources of stem cells include embryos, bone marrow, peripheral blood, umbilical cord blood, placental blood, and adipose tissue. Stem cells can be pluripotent, meaning that they are capable of generating most tissues on an organism. For example, pluripotent stem cells can give arise to cells of the skin, liver, blood, muscle, bone, etc. In contrast, multipotent or adult stem cells typically give rise to limited types of cells. For example, hematopoietic stem cells typically give rise to cells of the lymphoid, myeloid, and erythroid lineages. Viable cells are cells that are alive and frequently are capable of growth and division. Those of skill in the art are aware of methods to determine the viability of cells, e.g., by the ability to exclude trypan blue dye. The term stem cell as used herein includes progenitor cells unless otherwise noted.

“Nucleated cells” refers to cells that have a nucleus, i.e., an organelle that comprises chromosomal DNA. Nucleated cells include, e.g., white blood cells and stem cells. “Unnucleated cells” includes, e.g., adult red blood cells.

As used herein, the terms “plasma is substantially depleted” and “plasma-depleted” refer to the umbilical cord blood compositions of the present invention in which a volume of plasma greater than about 40%, 45%, 50%, 55%, 60%, 65%, 70%, 75%, 80%, 85%, 90%, or 95% has been removed. In a preferred embodiment, plasma is substantially depleted by centrifuging an umbilical cord blood-anticoagulant mixture and separating the cellular fraction from the plasma fraction. The plasma volume remaining following substantial depletion is typically from about 0% to about 30% by volume, preferably from about 10% to about 30% by volume.

The terms “non-red blood cell depleted” and “red blood cells are not depleted” as used herein refer to the umbilical cord blood compositions in which a volume of red blood cells less than about 30%, 25,%, 20%, 15%, 10%, 5%, 4%, 3%, 2%, or 1% has been removed. As used herein, the terms “red blood cell is substantially depleted” and “red blood cell-depleted” refer to processed umbilical cord blood units in which a volume of red blood cells greater than about 30%, 35%, 40%, 45%, 50%, 55%, 60%, 65%, 70%, 75%, 80%, 85%, 90%, or 95% has been removed. Although the present invention does not include a step of removing red blood cells from the umbilical cord blood unit, one skilled in the art will understand that the step of depleting the unit of plasma and/or any other processing steps may remove a small volume of red blood cells.

As used herein, the term “cryoprotectant” refers to an agent used to enhance the viability of cells while frozen. Cryoprotectants include, but are not limited to, dimethyl sulfoxide (DMSO), glycerol, ethylene glycol, propylene glycol, formamide, and hydroxyethyl starch (HES). Preferably, a low molecular weight polysaccharide such as dextran (e.g., Gentran 40) is added to the cryoprotectant mixture. In a preferred embodiment, the cryoprotectant solution comprises about a 10:1 ratio (volume/volume) of DMSO to Gentran 40 such as, e.g., 50% DMSO to 5% Gentran 40, which is added to the umbilical cord blood and anticoagulant mixture to provide a final concentration of from about 5% to about 10% DMSO.

The terms “proliferation” and “expansion” as used interchangeably herein with reference to cells, refer to an increase in the number of cells of the same type by division. The term “differentiation” refers to a developmental process whereby cells become specialized for a particular function, for example, where cells acquire one or more morphological characteristics and/or functions different from that of the initial cell type. Methods of cord blood stem cell expansion are known in the art. Such expansion techniques include those described in U.S. Pat. No. 7,399,633; WO/2013/086436, WO/2013/179633, US20180353541; Delaney et al., 2010, Nature Med. 16(2): 232-236; Zhang et al., 2008, Blood 111:3415-3423; and Himburg et al., 2010, Nature Med. 16, 475-482.

As used herein, “treating” or “treatment” refers to administration of a compound or agent or a composition to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

A “prophylactic treatment” includes a treatment administered to a subject who does not display signs or symptoms of a condition such that treatment is administered for the purpose of diminishing, preventing, or decreasing the risk of developing the condition. A “therapeutic treatment” includes a treatment administered to a subject who displays symptoms or signs of a condition and is administered to the subject for the purpose of reducing the severity or progression of the condition. A therapeutic treatment can also partially or completely resolve the condition.

An “effective amount” of an agent, e.g., a cell, is an amount sufficient to produce the desired effect, e.g., prevention or treatment of a disease or a symptom associated with the disease. A therapeutically effective amount of cells can vary according to the age and/or size of the individual, and the approximate volume of the ischemic area. The approximate volume and location of the ischemic area can be estimated, e.g., by serial magnetic resonance imaging images or computed tomography (CT) scanning.

As used herein, the term “administering” refers to the delivery of compositions of the present invention by any suitable route. Cells of the invention can be administered a number of ways including, but not limited to, parenteral (such term referring to intravenous and intra-arterial as well as other appropriate parenteral routes), intracoronary, intramyocardial, intrathecal, intraventricular, intraparenchymal (including into the spinal cord, brainstem or motor cortex), intracisternal, intracranial, intrastriatal, intranigral, intranasal, intraperitoneal, intramuscular, subcutaneous, intradermal, transdermal, or transmucosal administration, among others which term allows cells of the subject invention to migrate to the ultimate target site where needed. Preferably, patients are infused with one, two, three, or more umbilical cord blood units prepared according to the methods of the present invention. Multiple units such as double cord blood units can be administered simultaneously or consecutively (e.g., over the course of several minutes, hours, or days) to a patient.

Administration will often depend upon the disease or condition treated and may preferably be via a parenteral route, for example, intravenously, by administration into the cerebral spinal fluid or by direct administration into the affected tissue in the brain. In the case of stroke, the preferred route of administration will depend upon where the stroke is, but may be directly into the affected tissue (which may be readily determined using MRI or other imaging techniques), or may be administered systemically. In a preferred embodiment of the present invention, the route of administration for treating an individual post-stroke is systemic, via intravenous or intra-arterial administration.

Cells of the subject invention can be administered in the form of intact umbilical cord blood or a fraction thereof (such term including a mononuclear fraction thereof or a fraction of mononuclear cells, including a high concentration of stem or progenitor cells). The compositions according to the present invention may be used without treatment with a mobilization agent or differentiation agent (“untreated” i.e., without further treatment in order to promote differentiation of cells within the umbilical cord blood sample) or after treatment (“treated”) with a differentiation agent or other agent which causes certain stem and/or progenitor cells within the umbilical cord blood sample to differentiate into cells exhibiting a differentiated phenotype, such as a neuronal and/or glial phenotype.

The terms “grafting” and “transplanting” and “graft” and “transplantation” are used to describe the process by which cells are delivered to the site where the cells are intended to exhibit a favorable effect, such as repairing damage to a patient's central nervous system (which can reduce a cognitive or behavioral deficit caused by the damage), treating a neurodegenerative disease or treating the effects of nerve damage caused by stroke, cardiovascular disease, a heart attack or physical injury or trauma or genetic damage or environmental insult to the brain and/or spinal cord, caused by, for example, an accident or other activity. Cells can also be delivered in a remote area of the body by any mode of administration as described above, relying on cellular migration to the appropriate area to effect transplantation. Preferably, the cells can be administered with a blood brain barrier permeabilizer.

The term “neurodegenerative disease” is used herein to describe a disease which is caused by damage to the central nervous system and which damage can be reduced and/or alleviated by administration of cells described herein. Exemplary neurodegenerative diseases include Parkinson's disease, Huntington's disease, amyotrophic lateral sclerosis, Alzheimer's disease, Rett Syndrome, lysosomal storage diseases (“white matter disease” or glial/demyelination disease, as described, for example by Folkerth, J. Neuropath. Exp. Neuro., September 1999, 58:9), including Sanfilippo, Gaucher disease, Tay Sachs disease (beta hexosaminidase deficiency), other genetic diseases, multiple sclerosis, brain injury or trauma caused by ischemia, accidents, environmental insult, etc., spinal cord damage, ataxia and alcoholism. In addition, the present invention may be used to reduce and/or eliminate the effects on the central nervous system of a stroke or a heart attack in a patient, which is otherwise caused by lack of blood flow or ischemia to a site in the brain of said patient or which has occurred from physical injury to the brain and/or spinal cord. Neurodegenerative diseases also include neurodevelopmental disorders including for example, autism and related neurological diseases such as schizophrenia, among numerous others.

The term “therapeutic composition” or pharmaceutical composition” refers to the combination of an active agent with a carrier, inert or active, making the composition especially suitable for diagnostic or therapeutic use in vivo or ex vivo.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient) or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The term “subject” includes human and non-human animals. The preferred subject for treatment is a human. As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). In one embodiment, the subject is a human. In another embodiment, the subject is an experimental, non-human animal or animal suitable as a disease model.

The term “patient” is used herein to describe an animal, preferably a human, to whom treatment, including prophylactic treatment, with the cells according to the present invention, is provided. The term “donor” is used to describe an individual (animal, including a human) who or which donates umbilical cord blood or umbilical cord blood cells for use in a patient.

As used herein, the term “cardiomyopathy” has its conventional meaning as used in the art; that is, generally, the deterioration of the function of the myocardium (the muscle of the heart) for any reason. “Ischaemic cardiomyopathy” refers to a weakness in the muscle of the heart due to inadequate oxygen delivery to the myocardium, generally due to the absence or relative deficiency of its blood supply.

As used herein, the term “myocardium” has its conventional meaning as used in the art; that is, generally, the muscle of the heart. Direct administration of a composition comprising cells to the myocardium of the subject is envisaged by the present invention, meaning the composition is transferred from the device of administration (particularly envisaged, an injection catheter) to the myocardium tissue without having traversed any intervening tissue (for example coronary blood vessels). “Intramyocardial injection” refers to direct administration to the myocardium of the subject by injection.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Unless otherwise stated, the term “about” means within an acceptable error range for the particular value.

### EXAMPLES

### Example 1

This example illustrates a protocol for thawing and direct infusion of the plasma-depleted, cryopreserved cord blood units of the present invention without performing any washing steps. In this example and Examples 2-3, cord blood products from StemCyte are used.

### Sample/Specimen

StemCyte umbilical cord blood units cryopreserved with DMSO and stored at <-150° C.

### Reagents/Equipment/Supplies

37° C.±2° C. Water bath

Hemostats or Clamps

Sterile Water

Calibrated Thermometer used to measure temperature of 37° C. in the water bath

Large plastic bag, preferably sterile and sealable. (ZIPLOC, for example)

Liquid Nitrogen Storage Container

Alcohol Wipes

Multiple 60 cc Sterile Syringes

Sterile Wide Bore Needles (16 or 18 gauge)

10% Gentran 40 in 0.9% NaCl Injection 500 ml

Human serum albumin (HSA) 25% 12.5 g Albumin in 50 ml buffered diluent 50 ml.

### Calibration/Quality Control

The thaw procedure has been validated and shown to maintain sterility, viability, and minimum loss of TNC.

### Procedure

Before thawing

| Step | Action |
|---|---|
| 1 | Have hemostats or clamps ready and use to seal off any leaks immediately, if |
| | necessary. |
| 2 | Thaw the StemCyte cord blood at the patient's bedside, if possible. The patient |
| | should be pre-medicated according to the institutional practice at transplant center, |
| | taking into consideration the considerable DMSO (7.5 to 12.5 g) and free hemoglobin |
| | and potentially ABO/Rh incompatible red blood cells in the product. |
| 3 | Transport the cryopreserved cord blood to the patient's bedside in a validated liquid |
| | nitrogen storage container that will maintain the product at a temperature of <−150° C. |
| 4 | Before the cord blood product is thawed, verify the identity of the patient and the cord |
| | blood product. |
| 5 | Occasionally the StemCyte cord blood product may be in 2 freezing bags, or two cord |
| | blood products are being employed in a double product transplant. As the CFU's are |
| | stored at extremely low temperature, the bags are not as flexible as they were at |
| | ambient temperature. They are rigid and fragile and must be handled carefully. Use |
| | hemostats and clamps to seal off any leaks if occurred. |
| 6 | Only 1 bag should be thawed at a time. Infusion of the first bag should be completed |
| | with patient in a stable condition before thawing the subsequent bag. Likewise, |
| | infusion of the bag(s) of the first product should be completed with the patient in a |
| | stable condition prior to the thawing of the bag(s) of the second cord blood product. |
| 7 | Perform testing of the cord blood product using an attached segment or the residual |
| | left in the bag after the product has been infused. The testing should be performed as |
| | soon as the product is thawed by staff separate from the thawing and infusion |
| | personnel. This is especially important for time sensitive assays such as viability, |
| | which deteriorate rapidly after thawing and will give an underestimation of the true |
| | viability of the thawed cord blood product. |
| 8 | Once thawed, the cord blood product must be infused immediately. Do not refreeze |
| | the cord blood product once it has been thawed or partially thawed. |
| | Preparation of HSA/Gentran Rinse Solution |

(Note:
Prepare only if one plans to rinse the bag after infusion. Refer to “INFUSION” step below

| Step | Action |
|---|---|
| 1 | Remove the cap and clean the rubber diaphragm of a 50 ml vial of HSA with an |
| | alcohol prep (other institutional procedures approved for surface disinfection may be |
| | substituted). |
| 2 | Draw 10 ml of HSA into a 60 CC syringe. |
| 3 | Draw 40 ml of Gentran into a separate 60 CC syringe. |
| 4 | Empty contents of HSA syringe into a transfer bag through one of the ports. |
| 5 | Empty contents of Gentran syringe into a transfer bag through one of the ports. |
| 6 | Mix the contents thoroughly. |
| 7 | The final volume should be 50 ml with a final concentration of 5% for HSA and 80% |
| | for Gentran. |
| 8 | Label bag with the date and time that the rinse solution was prepared according to |
| | institutional procedures. |

Thawing Of the Cord Blood Product

| Step | Action |
|---|---|
| 1 | Fill water bath with enough sterile water to completely immerse the umbilical cord |
| | blood product. |
| 2 | Allow the water in the water bath to equilibrate to 37° C. ± 2° C. The water in the |
| | water bath must maintain this temperature throughout the thaw procedure. |
| 3 | Have a plastic bag (preferably sterile) easily accessible to the water bath. This bag is |
| | used to salvage the cord blood product in case of an unlikely event that leakage is |
| | detected because of compromise of integrity of the freezing bag containing the cord |
| | blood product. |
| 4 | Carefully remove the cryopreserved cord blood product from the metal cassette and |
| | verify the identification of the product and inspect the bag for any breakage. If |
| | breakage is observed, note the appearance and pattern of breakage in the enclosed |
| | initial outcome data form 200T. If a second bag or product is in the liquid nitrogen |
| | transport container, close the lid as soon as the first product is removed to ensure |
| | maintenance of proper temperature (<−150° C.) for the second bag/product. |
| 5 | Place the cryopreserved cord blood product into the plastic bag in order to prevent the |
| | cord blood product from direct contact with the water. This step should be performed |
| | as soon as the product is removed from the liquid nitrogen and after verification of the |
| | identity and inspection of the product. For unsealed bags, gently knead the cord |
| | blood from the outside of the plastic bag, being careful not to get water into the outer |
| | bag. If breakage of the freezing bag is observed, use hemostats or clamps to seal off |
| | the leaking area. It is critical that contamination is minimized since contents of the |
| | bag will likely leak into the outside plastic bag. |
| 6 | For sealed bags, thaw the cord blood product by sealing the bag after expressing most |
| | of the air, immersing it in the water, and gently kneading the cord blood product |
| | through the plastic bag. |
| 7 | Do not leave the cord blood product unattended at any time during the thaw |
| | procedure. |
| 8 | Check the cord blood bag for leaks as it thaws. If leaks are observed, follow |
| | institutional protocols for infusion of products with breaks or leaks. Special |
| | monitoring and prophylaxis antimicrobial treatment of the patients may be required. |
| | Do NOT discard the cord blood. |
| 9 | Remove the cord blood product from the water bath as soon as it is in an “icy slushy” |
| | state. Remove the freezing bag from the outer plastic bag and disinfect the infusion |
| | ports with alcohol wipes (or other approved institutional procedure for surface |
| | disinfection). The thawing step should not take longer than 5 minutes. |

Infusion

| Step | Action |
|---|---|
| 1 | Immediately draw up the product in one or more sterile 60 cc syringes through one of |
| | the disinfected ports of the freezing bag using the widest bore needle possible to |
| | minimize cell shearing. |
| | If there was leakage into the outer plastic bag, carefully remove the freezing bag from |
| | the outer bag, draw up any remaining product left in the freezing bag through the |
| | disinfected infusion port. Then carefully draw up the leaked product from the outer |
| | plastic bag. |
| 2 | The product in the syringe is then immediately IV pushed through a central line, with |
| | a T connection, as rapidly as possible (5-10 ml/min). Because of the viscosity of the |
| | StemCyte cord blood product, considerable resistance may be expected. |
| 3 | The freezing bag can be rinsed with 10 to 20 cc of a 80% Dextran/5% Human Serum |
| | Albumin rinse solution, and can then be drawn up again and IV pushed through the |
| | central line. The entire product should be, ideally, infused within 10 minutes of |
| | thawing. Infusion should be initiated no more than 20 minutes from the initiation of |
| | thawing. |
| 4 | Thaw the second cord blood bag, if applicable, only after the first bag has been |
| | completely infused. |

Samples of cryopreserved plasma depletion cord blood products were assayed to determine cytokine profiles using a R&D Human XL Cytokine Discovery 14 Plex panel. The results are shown in Table 1A above. The results showed a level of anti-inflammatory cytokine, IL-10, was significantly higher than that of pro-inflammatory cytokine, such as IL-1-beta, IL-2, IL-6, IFN-gamma and TNF-alpha. In addition, relatively higher levels of GFs, EGF, FGF-basic, VEGF, G-CSF, and GM-CSF were observed in comparison with those of cytokines, IL-1-beta, IL-2, IL-4, IL-5, IL-6, IFN-gamma and TNF-alpha. Since high amounts of EGF, VEGF, G-CSF, and IL-10 were detected in PD CB products, the infusion of PD CB products may not only restore immune homeostasis but also facilitate brain repairing in acute stroke patients.

### Example 2

This example illustrates designs and protocols for a phase 1 clinic study in connection with allogeneic PD-UCB infusion for adults with acute ischemic stroke. This is a multicenter study in patients aged 45-80 years who sustain acute stroke without t-PA therapy. A total of 6 subjects are enrolled according to the inclusion and exclusion criteria described below. Subjects are given a series of baseline neurological assessments, blood tests, and MRI. Umbilical cord blood units are selected from a public cord blood bank based on ABO/Rh blood type matched, HLA matched, and cell dose, targeting a range of 0.5 to 5×10′ total nucleated cells/kg.

Plasma depleted processed Umbilical cord blood from StemCyte Taiwan is administered intravenously as a single infusion between 3 to 10 days post-stroke. Subjects are monitored for 6 hours post-infusion, and followed up 24 hours later. Subsequent follow up phone calls occur at 1, 6, and 12 months, including telephone surveys on post-stroke rehabilitation and functioning. A follow up clinic visit at 90 days include a neurological exam, MRI, and blood tests.

### Selection of Cord Blood Unit

A search is carried out upon receipt of the subject's information including HLA typing from the transplant center. A Summary Search Report for Acute Stroke Clinical Trial is created accordingly to include candidate CB units which meet the following criteria: (1) at the least of 4/6 match to the subject on low resolution HLA typing; (2) identical ABO/Rh blood type between subject and donor, and (3) the total mononucleated cells (TNCs) count is between 2 to 5×10⁸.

UCB units PD-UCB units were obtained from Stemcyte. The units were screened and tested negative for HIV-½, HTLV-I/II, HCV, HBc, CMV IgM+ IgG, syphilis RPR, HBSAg, and WNV. If needed, UCB cells were selected to match for ABO/Rh or HLA of a subject. A single IV infusion was used to administer allogenic UCB to the subject. After the administration, the subject was administered with 20% mannitol 200 ml i.v. for 30±10 min twice, which were separated by 8±2 hours. Alternatively, the subject was administered with 20% mannitol 100 ml/60 minutes i.v. q4 h. Both were effective to permeabilize the blood brain barrier.

### Subjects

This study of UCB infusion for adults with ischemic stroke was a multisite, phase I exploratory clinical trial studying the safety and feasibility of a single allogeneic UCB i.v. infusion into 6 adults who had each experienced an ischemic stroke. This study was conducted at Hualien Tzu Chi Hospital, Buddhist Tzu Chi Medical Foundation, Taiwan, Republic of China. The study was approved by each clinical site's local institutional review board.

Inclusion Criteria:

| | |
|---|---|
| 1. | Age between 45 and 80. |
| 2. | Acute ischemic stroke without t-PA therapy. |
| 3. | Brain MRI shows acute brain ischemic stroke in the MCA territory |
| | exclude hemorrhagic stroke. No mid line shift; no hemorrhagic |
| | transformation. |
| 4. | NIHSS Scale: 8 to 16 |
| 5. | Check NIHSS 2 times, 2^{nd} score cannot be decrease 4 points compare |
| | with 1^{st} score. |
| 6. | HLA matched ≥ 4/6 with ABO/Rh matched. |
| 7. | The cord blood will be administered once intravenously no later |
| | than 10 days after stroke. |

Exclusion criteria
Medical Conditions

| | |
|---|---|
| 1. | Has a medical history of neurological or orthopedic pathology with a deficit as |
| | a consequence that results in a modified Rankin Scale >1 before stroke or has a pre- |
| | existing cognitive deficit. |
| 2. | Has clinically significant and/or symptomatic hemorrhage associated with |
| | stroke. |
| 3. | Has new intracranial hemorrhage, edema, or mass effect that may place patient |
| | at increased risk for secondary deterioration when assessed prior to infusion. |
| 4. | Has hypotension as defined as the need for IV pressor support of systolic |
| | blood pressure < 90. |
| 5. | Has isolated brain stem stroke. |
| 6. | Has pure lacunar stroke. |
| 7. | Requires mechanical ventilation. |
| 8. | Requires a craniotomy. |
| 9. | Has a serious psychiatric or neurological disease which could alter |
| | evaluation on functional or cognitive scales. |
| 10. | Has an active systemic infection or is HIV positive. |
| 11. | Has had an active malignancy within 3 years prior to the start of screening |
| | excluding skin cancers other than melanoma. |
| 12. | Has known coagulopathy such as Factor V Leyden, AntiPhospholipid |
| | Syndrome (APC), Protein C, Protein S deficiency, sickle cell, anticardiolipin antibody, |
| | or phospholipid syndrome. |
| 13. | Has any concurrent illness or condition that in the opinion of the investigator |
| | might interfere with treatment or evaluation of safety. |
| 14. | Has current or recent history of alcohol or drug abuse, or stroke associated |
| | with drug abuse. |
| 15. | Pregnant as documented by urine or blood test. |

Concomitant or Prior Therapies

| | |
|---|---|
| 1. | Subjects currently receiving immunosuppressant drugs. |
| 2. | History of prior transfusion reaction. |
| 3. | Currently on dialysis. |
| 4. | Recipient of bone marrow or organ transplant. |
| 5. | Renal insufficiency with serum creatinine >2.0 mg/dL. |
| 6. | Hepatic insufficiency (bilirubin > 2.5 mg/dL or transaminases > 5x the upper limit |
| | of normal). Patients with Gilberts syndrome are eligible for study enrollment if |
| | other liver function tests are normal, regardless of bilirubin level. |
| 7. | Any previous or current treatment with angiogenic growth factors, cytokines, |
| | gene or stem cell therapy. |
| 8. | Subjects participating in another interventional clinical trial of an investigational |
| | therapy within 30 days of screening. |

Other

| | |
|---|---|
| 1. | Pregnant or lactating women. |
| 2. | Unable to be evaluated for follow up visits. |

### Test Period and Progress

Each subject in the trial is tracked for a total of 12 months from the time of beginning to the end of the trial.

### Study Design and Procedures

Cell Preparation and Operation:

As once thawed the cord blood cell activity begins to decrease, the thawing process of cord blood must be carried out at the hospital site. A fluid warmer used in healthcare facilities for warming fluids or blood product can be used to thaw the cells.

Clinical Procedures

This study is a Phase I study conducted in a case-by-case manner. Six subjects are admitted for this study. For a subject exhibiting limb hemiplegia, UCB w administered at day 9 after onset of stroke. A single IV infusion is used to administer allogenic UCB (containing MNC 2 to 5×10⁸) to the subject. After the administration, the subject is administered with 20% mannitol 200 ml i.v. for 30±10 min twice, which are separated by 8±2 hours. Alternatively, the subject is administered with 20% mannitol 100 ml/60 minutes i.v. q4 h.

The following are procedures for participating in the trial and routine examination.
1. Basic information, history of health illnesses and history of medication (traced back to three months prior to admission to the study).
2. Vital sign measurements.
3. Physical examinations.
4. Blood tests for the following:
   1) Tissue typing HLA-HLA-ABC multiple antigen, Tissue typing HLA-HLA-DR multiple antigen, and ABO blood group test (A, B, AB, 0 blood grouping, RH(D).
   2) CBC-I (WBC, RBC, Hb, Hct, Platelet count, MCV, MCH, MCHC) and WBC differential count.
   3) APTT (activated partial thromboplastin time), Prothrombin time, and Prothrombin Time and International Normalized Ratio (INR).
   4) Blood biochemical examination, including Na⁺ (Sodium), K⁺ (Potassium), and Cl⁺ (Chloride) concentrations, blood sugar levels (ac or pc), serum glutamate-oxaloacetate transaminase (S-GOT), serum glutamate-pyruvate transaminase (SGPT), blood urea nitrogen (BUN), Creatinine (B) CRTN.
   5) Erythrocyte sedimentation rate (E.S.R), C.R.P (C-reactive protein)-Nephelometry.
   6) Cytokines, including IL-2, IL-6, IL-10, IL-17, IFN-γ, and TNF-α.
5. Glasgow Coma Scale (GCS).
6. Electrocardiogram (EKG).
7. Chest X-tray.
8. Brain MRI.
9. Abdominal sonography.
10. NIHSS, Berg Balance Scale (BBS), and Barthel Index (BI).

Visit 1: Screening (the following activities are carried out in hospital):

A subject is examined to obtain the ten categories of information described above. Among them, the NUBS is obtained twice separated by 24±1 hrs to establish baseline/baseline.

Visit 2: Human umbilical cord blood infusion (Day 0)

1. Confirm that the subject's consent form is signed.

2. According to tissue typing HLA-HLA-ABC multiple antigen, tissue typing HLA-HLA-DR multiple antigen, and ABO blood group test (A, B, AB, 0 blood grouping, and RH (D)), identify a matched cord blood unit. The matched, frozen cord blood unit is sent to the hospital.

3. Monitor the vital signs of the subject before infusion.

4. Thaw the frozen cord blood unit in the in-hospital laboratory according to the procedure described above.

5. The umbilical cord blood (containing about 2-5×10⁸ monocytes) is injected intravenously for one injection. Mannitol (20% mannitol, 200 ml I.V. for 30±10 min) is given to the subject with two injections (an interval of 8 hours ±2 hrs). After the administration, vital signs are monitored as described below:
. 4 times at every 15±5 minutes after the start of infusion;
. 1 time after the first mannitol injection;
. 1 time just before the second mannitol injection;
. 4 times after the second mannitol injection every 6 hours ±30 minutes.

Visit 3: Recovery Period (24+8 hours after UCB infusion)

At 24±8 hours after UCB infusion, the subject is checked for the following:
1. Vital signs;
2. GCS;
3. Blood tests for the following:
   1) CBC-I (WBC, RBC, Hb, Hct, Platelet count, MCV, MCH, MCHC) and WBC differential count;
   3) APTT (activated partial thromboplastin time), Prothrombin time, and Prothrombin Time and International Normalized Ratio (INR);
   4) Blood biochemical examination, including Na+(Sodium), K⁺ (Potassium), and Cr (Chloride) concentrations, blood sugar levels (ac or pc), serum glutamate-oxaloacetate transaminase (S-GOT), serum glutamate-pyruvate transaminase (SGPT), blood urea nitrogen (BUN), Creatinine (B) CRTN;
   5) Erythrocyte sedimentation rate (E.S.R), C.R.P (C-reactive protein)-Nephelometry; and
   6) Cytokines, including IL-2, IL-6, IL-10, IL-17, IFN-γ, and TNF-α.
4. Brain MRI;
5. Abdominal sonography;
6. NIHSS, Berg Balance Scale (BBS), and Barthel Index (BI);
7. Observe and record adverse events, assess whether the subject has GVHD;
8. Review medication records; and
9. Review the subject's medical records and record the data anonymously for research use.

Visit 4: Recovery Period (48+8 hours after UCB infusion)

At 48±8 hours after UCB infusion, the subject is examined again for items 1-3 and 6-9 under Visit 3.

Visit 5: Recovery Period (72+8 hours after UCB infusion)

At 72±8 hours after UCB infusion, the subject is examined again for items 1-3 and 5-9 under Visit 3.

Visit 6: Recovery Period (7+1 days after UCB infusion or one day before discharge)

The subject is examined for vital signs, GCS, NIHSS, BBS, BI, adverse events (including GVHD). The subject's medication and medical records are also reviewed.

Visit 7: Observation Period (1 month+7 days after UCB infusion)

The subject is examined for vital signs, brain MRI, NUBS, BBS, BI, adverse events (including GVHD). The subject's medication and medical records are reviewed and recorded.

Visits 8, 9, 10, and 11: Observation Period (3, 6, 9, 12 months+7 days after UCB infusion):

The subject is examined for vital signs, blood tests in the same manner as those for visit 3, abdominal sonography (3 and 12 months ±7 days after UCB infusion), brain MRI (6 and 12 months ±7 days after UCB infusion), NIHSS, BBS, BI, and adverse events (including GVHD). The subject's medication and medical records are reviewed and recorded.

### Example 3

This example illustrates that allogenic UCB units that have been substantially depleted of plasma, but not depleted of red blood cells are safe and effective for an adult patient with acute ischemic stroke.

### Patient Characteristics:

1. Age: 46
2. Gender: male
3. On set time of stroke: 6/3/2019, 9 pm
4. NIHSS: baseline 8; after 24 hours 9
5. Signed up ICF at 2019/06/06 enrolled to acute stroke study
6. Dx: right MCA/ACA infarction with left hemiplegia

Other Physical history: Gastric ulcer, Hypertension, Chronic kidney disease (CKD) with end-stage renal disease (ESRD) under hemodialysis (HD) QW1, 3, 5 since 2011, angina, and Hyper parathroidism s/p partial parathyroidectomy.

Accoridng to the patient's HLA type, a matching UCB unit was identified with the following UCB Profile:
1. TW-02-04191;
2. Collected on Jul. 22, 2002 by StemCyte Taiwan
3. Stored for 17 yrs before infusion;
4. Processed by Plasma Depletion Method;
5. HLA Match=6/6;
6. Pre-freeze TNC=65.28×10⁷; and
7. Pre-freeze CD34=105.8×10⁴.

The patient was administered with the UCB (2.63×10⁸) in the manner described above at day 8 post stroke. The UCB infusion started at 12:47 am and ended at 13:13 pm. The patient's condition was stable during UCB infusion. After a 30-minute break, the patient was administered with 4 doses of 20% 100 ml mannitol intravenous injection every 4 hours (mannitol 100 ml/60 minutes i.v. q4 h).

At 24 hours after the UCB infusion (about 9 days after onset of stroke), the patient was examined in the manner described above. The brain MRI indicated that with the time passing by, the edema progressed to maxima.

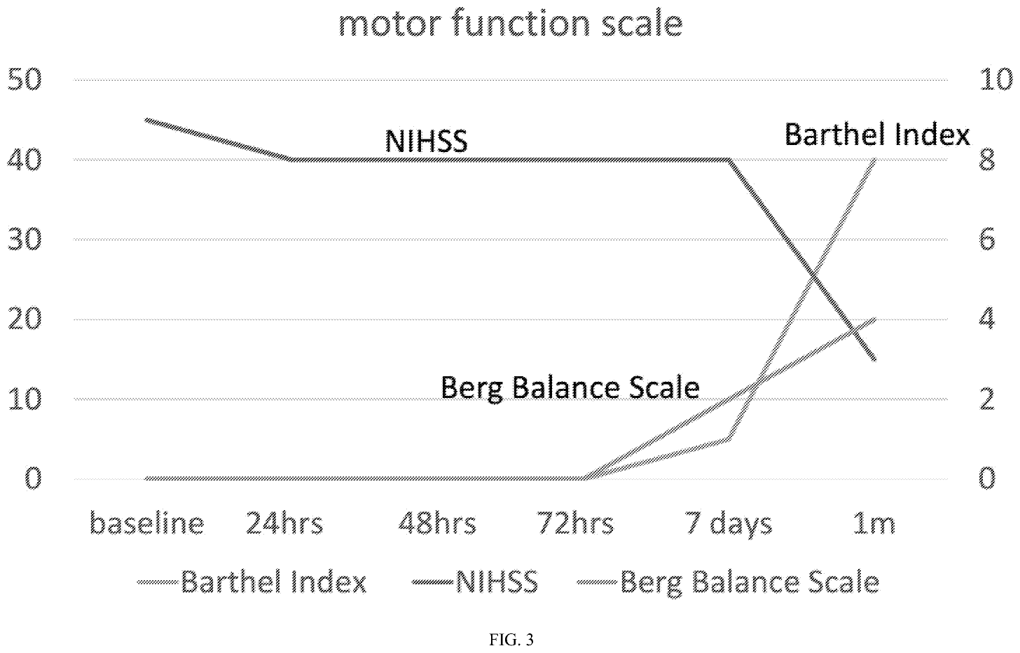

At 7-14 days after UCB infusion, the patient showed motor function improvement. At 1 month after UCB infusion, the patient showed further and more significant motor function improvement. The related NIHSS, BI, and BBS changes during this period are shown in FIG. 3. In particular, at Day 0, before infusion of umbilical cord blood, the patent exhibited left side paralysis. At Day 7, 7 days after infusion of UCB, the patient's left fingers were movable. At 1 month after infusion of UCB, the patient could move left hand/fingers and leg, and make a fist. His NIHSS decreased from 9 to 3.5.

The above results demonstrated significant recovery for the adult patient with ischemic stroke within a short period of time after receiving allogeneic umbilical cord blood with HLA 6/6 matching processed by Plasma Depletion Method. Such recovery is unprecedented and has not observed in other clinical trials.

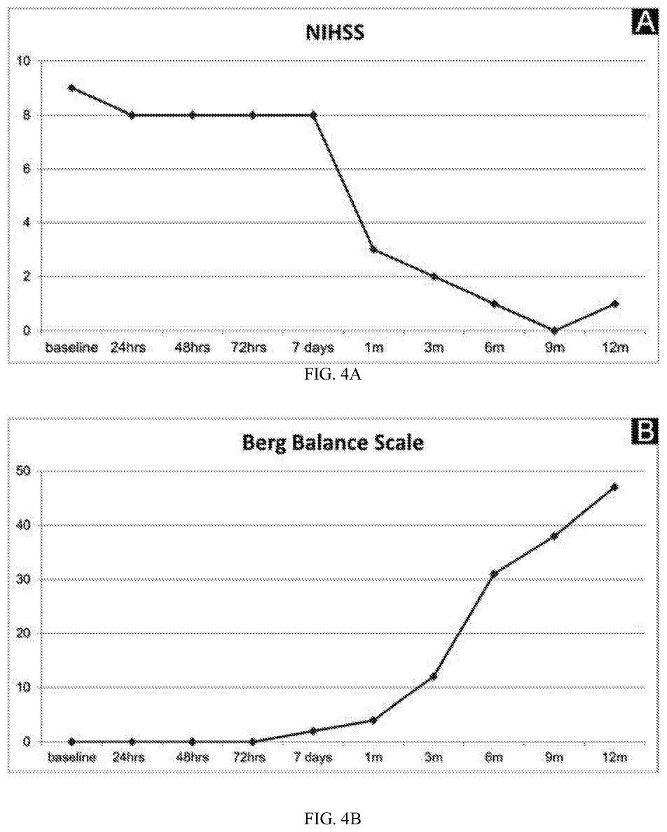

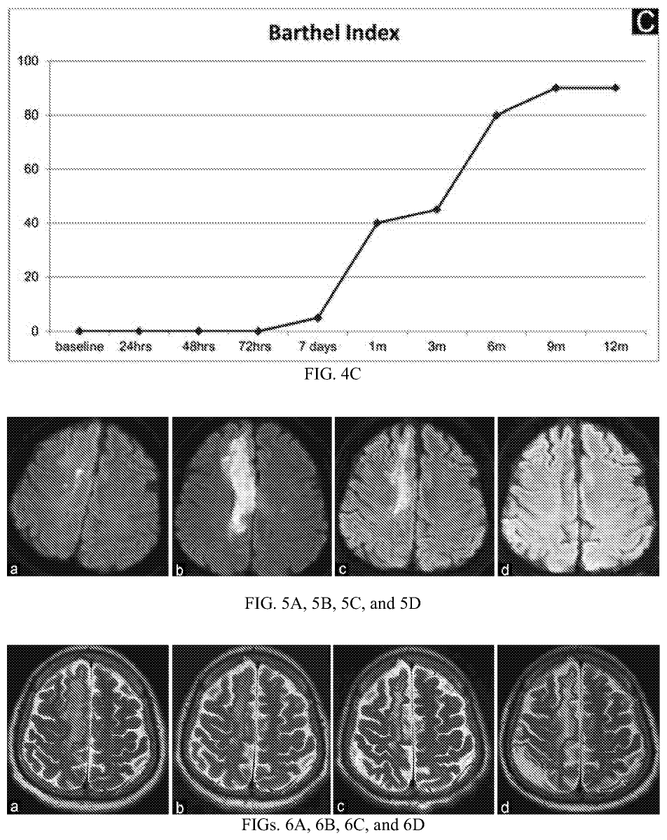

The patient was discharged 8 days after CB transfusion and followed up at 1, 3, 6, and 12 months in the manner described above for NIHS, neurological function and MRI checked up. It was found that patient's neurological functions improved gradually after cord blood transfusion. His NUBS improved from 9 point at baseline to 1 point at 12 months post-CB transfusion (FIG. 4A), Berg balance score from 0 point to 48 point (FIG. 4B), and Barthel index score from 0 point to 90 point (FIG. 4C). No splenomegaly was found through 12 observations by abdominal ultrasound examination. Two adverse events including insomnia and upper respiratory tract infection developed and were treated with medication successfully. No serious adverse event was observed. Diffusion weighted imaging (DWI) 2 hours and 8 days after stroke revealed infarction on right corona radiata (increased white intensity), which scattered 3 months later and disappeared 6 months later (FIGS. 5A-5D). T2 weighted images detected 2 hours, 8 days, 3 months and 6 months after infarction shown increased white intensity on right corona radiata (FIGS. 6A-6D).

Efficacy of cell therapy for cerebral stroke depends on factors including cell type, cell origin, cell number, timing of cell treatment, and route of cell delivery. The time window for repairing of damage neural cells or renew of neural cells after the initiation of stroke is short, e.g., within 72 hours based on animal experiment. Early cell therapy in acute or subacute stage of stroke might result in better outcome. The above-mentioned patient underwent a subclinical 2^{nd} stroke identified from MRI examination 3 months after stroke. The T2 images shown loss of brain parenchyma and CSF acumination only in the region of 2^{nd} stroke but not in the 1^{st} stroke region which has been treated by cell therapy in acute stage (FIGS. 6C and 6D).

CB cells exhibit better therapeutic potential in acute and subacute stage of stroke. CB cells can protect the penumbral tissue from the further injure induced by inflammation reaction post stroke, because they possess immunomodulatory and anti-inflammatory effects in addition to regenerative effect. CB cells can also exert their immunomodulatory activity by mean of changing the phenotype of splenocytes. In acute or subacute stages, CB therapy by intravenous delivery can achieve neural protection thought modification of systemic immunomodulation.

The benefits of mesenchymal stem cells (MSCs) therapy are achieved and expanded in laboratory. The safety of MSCs has been documented in many clinical tries, however the efficacy is unclear. The quality, activity, and developmental potential of donor stem cells are important for successful stem cell therapy. The cell conditions are much dependent on the origin of donor. In autologous cell transplantation, donor cells retrieved from patients having a disorder may not be have satisfactory quality, activity, or developmental potential. Unlike autologous transplantation, cryopreserved HUCMB for allogenic transplantation from healthy donor provides an alternative that has much better stability and quality.

An appropriate number of stem cells for stroke therapy can be determined by a person skilled in the art. The minimal acquirement for intravenous injection of MSCs was recommended at a single dose of 840 million (Borlongan CV et al. Stem Cells Translational Medicine. 2019; 8(9):983-8). One skilled in the art will appreciate that the risk-and-benefit consideration of cell number and toxicity should be the maximal benefit and smallest cell dosage (Sarmah D, et al., Translational stroke research. 2018; 9(4):356-74). To enhance the permeability of stem cell to across the BBB may help reduce the amount stem cells. To that end, mannitol can be used to break the BBB to facilitate the peripheral delivery of stem cells. Although the cell amount of this reported case was about 263 million, mannitol was used to open the BBB 30 minutes after cell infusion. Besides enhancing permeability of stem cells, mannitol could enhance the neurotrophic factors and neural growth factors to across the BBB too. Therefore, the method described herein allows one to take advantages of the beneficual effects from transplanted CB cells themsevles and others, including synaptogenesis, porliferation of immature neurons, and migration of neuronal cells in addition to regeneration of CB cells.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method of treating or ameliorating a cardiovascular disease or brain injury comprising
identifying a subject in need thereof, and
administering to the subject an effective amount of a therapeutic composition comprising umbilical cord blood (UCB).

2. The method of claim 1, wherein the therapeutic composition comprises plasma-depleted (PD) UCB or red cell-reduced (RCR) UCB.

3. The method of claim 1, wherein the therapeutic composition further comprises a cryoprotectant.

4. The method of claim 3, wherein the cryoprotectant is dimethyl sulfoxide (DMSO).

5. The method of claim 2, wherein the PD UCB is not depleted in red blood cells when compared to whole blood UCB.

6. (canceled)

7. (canceled)

8. The method of claim 1, wherein the therapeutic composition is obtained by thawing a stored composition comprising UCB.

9. (canceled)

10. The method of claim 8, wherein the step of thawing comprises incubating the stored composition in a bath maintained at between about 37° C.±2° C.

11. The method of claim 8, wherein the stored composition is not washed after thawing and is administered as the therapeutic composition to the subject.

12. The method of claim 8, wherein the step of administering is completed within 1 to 2 hours after the thawing is completed.

13. The method of claim 1, wherein the UCB comprises mononucleated cells and the mononucleated cells are administered to the subject at approximately 2-5×10⁸ mononucleated cells/kg to approximately 1×10⁸ cells/kg.

14. The method of claim 1, wherein the therapeutic composition is administered by infusion.

15. (canceled)

16. The method of claim 1, further comprising administering a blood-brain barrier (BBB) permeabilizer composition to the subject.

17. The method of claim 16, wherein the BBB permeabilizer composition comprises mannitol.

18. The method of claim 1, wherein the cardiovascular disease is a stroke or cardiomyopathy.

19. (canceled)

20. The method of claim 18, wherein before the administering step the subject has a National Institutes of Health Stroke Scale (NIHSS) score of 4 to 32 or higher.

21. The method of claim 1, wherein the subject has larger than 4/6 HLA match.

22. (canceled)

23. The method of claim 1, wherein the method further comprises administering the subject an immunosuppression agent.

24. The method of claim 1, wherein the subject has not administered with a fibrinolytic drug before the administering step.

25. The method of claim 24, wherein the fibrinolytic drug is tissue plasminogen activator (TPA).

26. The method claim 18, wherein cardiomyopathy is ischemic cardiomyopathy.
